(19) United States
(12) Patent Application Publication
(10) Pub. No.: US 2002/0091355 A1
(43) Pub. Date: Jul. 11, 2002

(54) Compliant delivery catheter

(75) Inventor: Scott William Hayden, Minneapolis, MN (US)

Correspondence Address: Scott Hayden, Ascend Medical, Inc., 3449 34th Ave. S., Minneapolis, MN, 55406, US

(73) Assignee: Ascend Medical, Inc., Minneapolis, MN

(21) Appl. No.: 10/037,857

(22) Filed: Jan. 7, 2002

Related U.S. Application Data
(60) Provisional application No. 60/249,809, filed on Nov. 17, 2000.

(51) Int. Cl. A61M 29/00

(52) U.S. Cl. 604/104; 606/191

(57) ABSTRACT

Several embodiments of a compliant catheter and methods of use are disclosed. The distal portion of the catheter maintains a collapsed position during navigation through a vessel and may be expanded upon reaching the target location. The collapsed position allows better maneuverability in small and tortuous or heavily diseased vessels. Expanding the distal portion of the catheter allows better device passage and increased fluid flow. This catheter may be used for device or fluid delivery, or for removing fluid and/or solids from a vessel or duct.

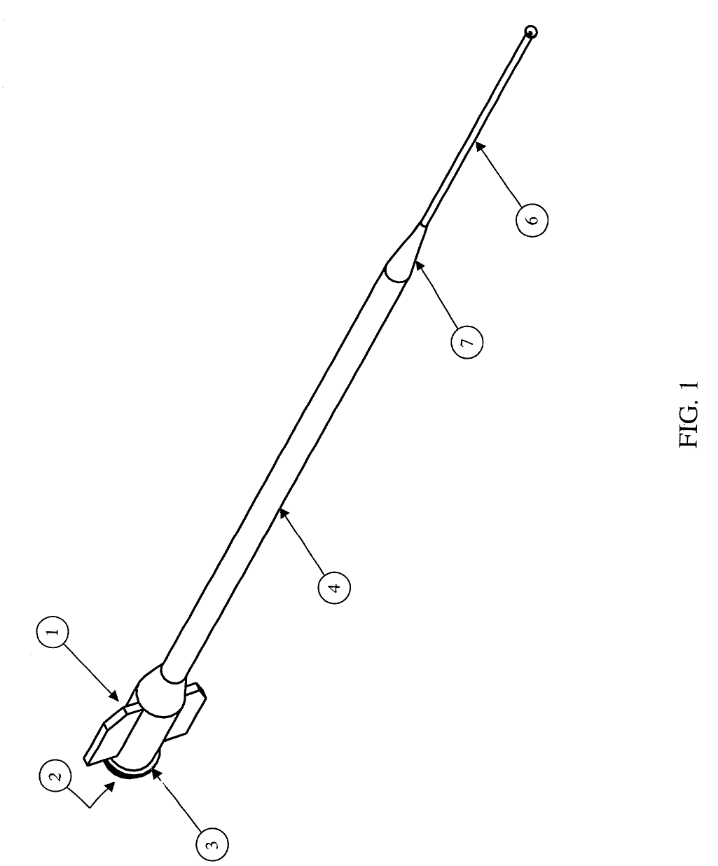

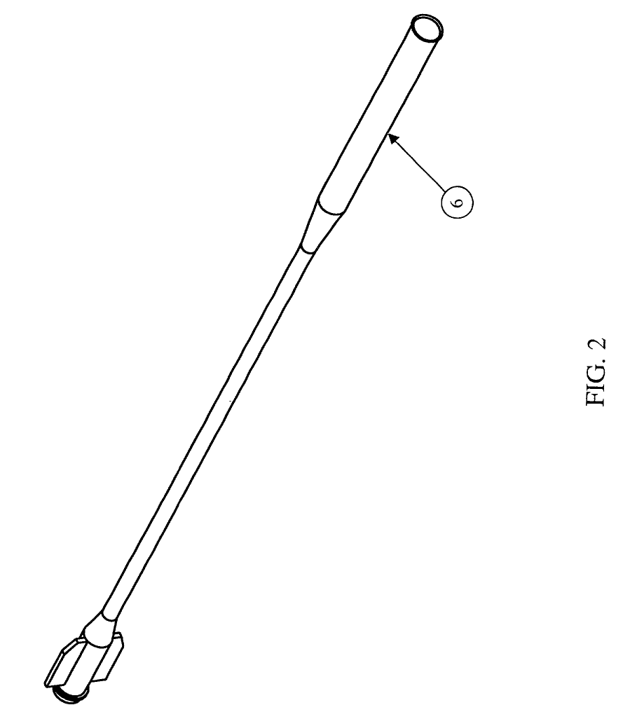

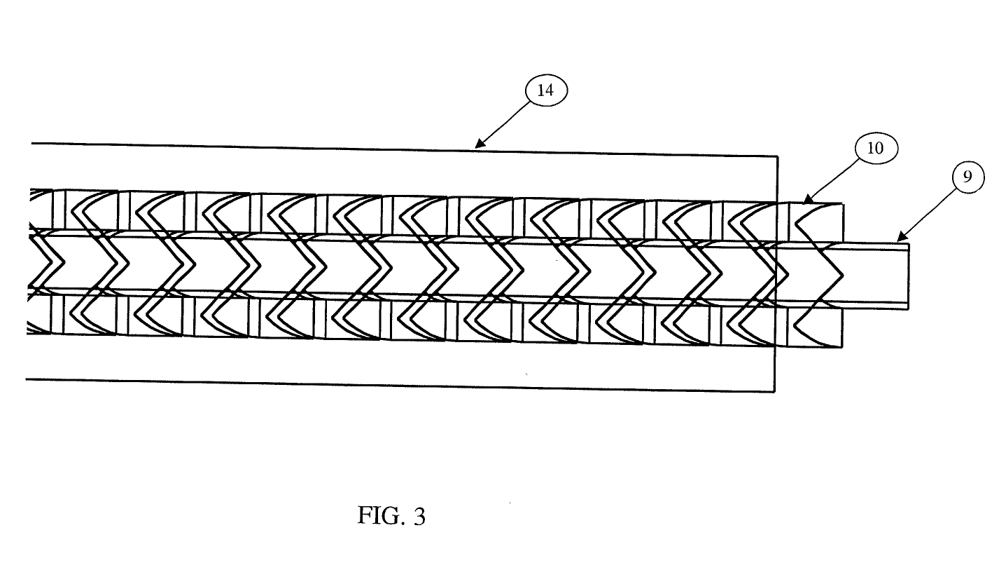

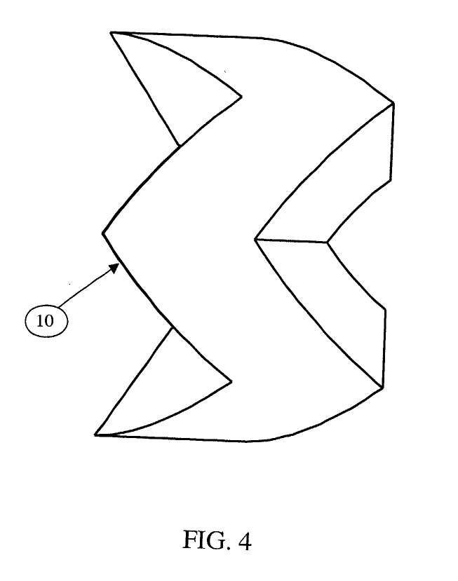

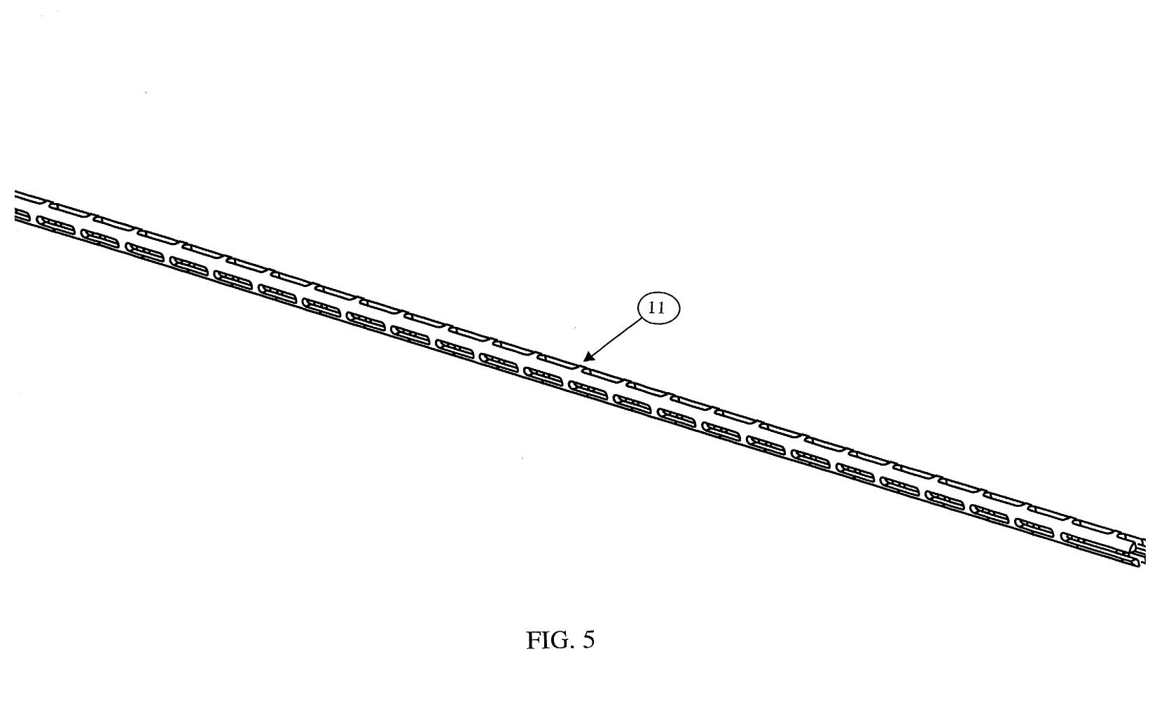

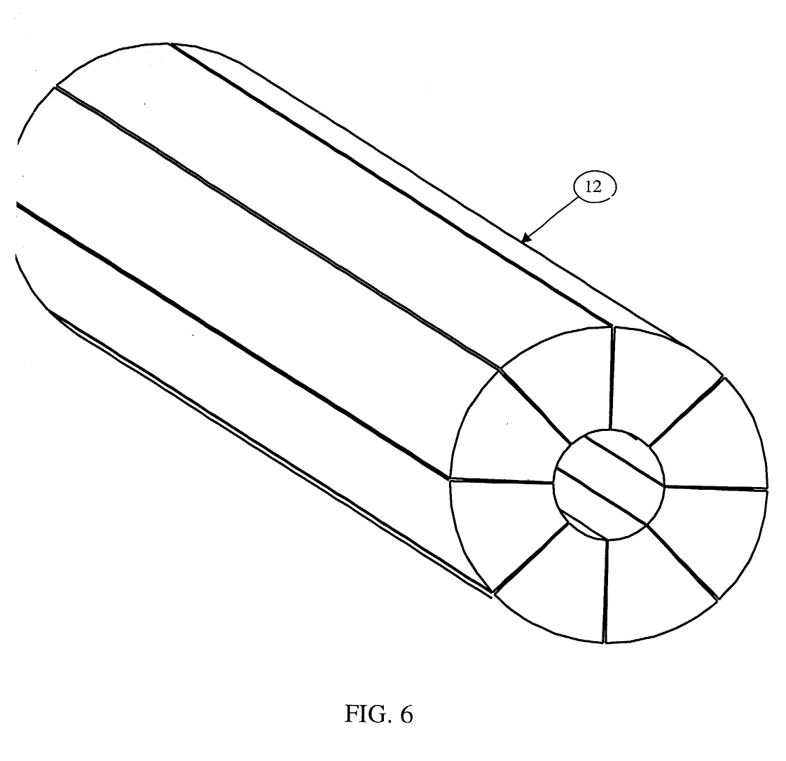

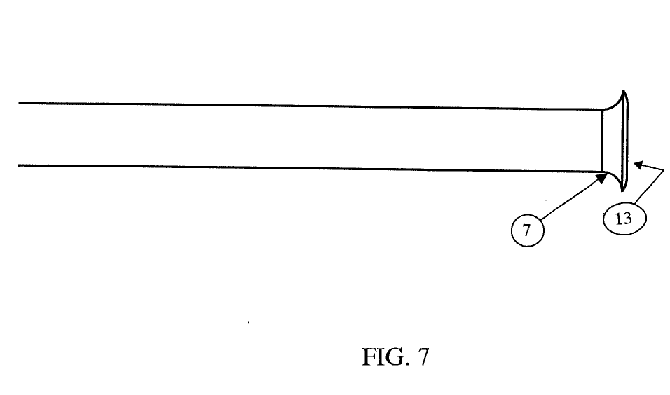

### FIELD OF THE INVENTION

This invention is a medical device. More specifically, it is a catheter-type assembly having an expandable distal section maneuverable with or without use of a steerable guide wire.

### BACKGROUND OF THE INVENTION

Percutaneous catheterization is a type of medical treatment that is generally less invasive than directly accessing an internal body site for treatment, such as when using general surgery methods. In catheterization techniques, a long small diameter wire and long tubular catheter are typically introduced into the body through a puncture site in the arm or leg. It is then passed to target site, usually through passageways such as the vascular tree. Treatment or diagnostic procedures may then be performed using the catheter by manipulation of the portion of the catheter remaining outside the body or by a separate external steering device, such as an external magnet.

This invention involves a catheter-type delivery system with two distinct sections—the first portion is a non-compliant shaft and the second portion comprising of a collapsible/expandable catheter shaft. The catheter takes the general form of an elongated medical device with the advantageous features of a guidewire for its maneuverability and relatively small external distal diameter and a delivery catheter for its relatively large and lubricious inner lumen. Often the target site which one wishes to access by catheter is buried within a soft tissue, such as brain, heart, arms, legs or liver, and is only reached by a tortuous route through small vessels or ducts—typically less than about 3 mm lumen diameter—in the tissue. The difficulty in accessing such regions is that the catheter must be quite flexible, in order to follow the tortuous path into the tissue, and at the same time, stiff enough to allow the distal end of the catheter to be manipulated from an external access site, which may be as much as a meter or more from the tissue site. The advantages features of this device allow access to small, tortuous, and/or heavily diseased vessels or ducts and provide less occlusion of the vessel or duct during a procedure.

Other medical devices to be compared to this invention include perfusion guide wires and delivery catheters.

### Perfusion Guidewires

Various medical procedures require fluids to be delivered to specific locations within the body, typically via a fluid delivery catheter. A narrow steerable guidewire is often used to maneuver through narrow, tortuous, and/or branching body passageways. After the guidewire has been directed to the desired location, a fluid delivery catheter may be inserted over the guidewire. The guidewire is usually removed before fluid delivery begins. Guidewires that are themselves capable of fluid delivery are also known in the art.

Some medical conditions may require a guide wire for fluid delivery catheter to facilitate treatment. Delivery of drugs such as thrombolytic agents, or radiopaque dye for visualization, may be desirable at a target site in small and/or tortuous vessels, or heavily diseased vessels. A catheter may be unable to access such a site due to its large diameter and/or its limited pushability, steerability and kink resistance.

The fluid delivery guidewire system takes the general form of an elongated medical device guidewire and thus enjoys the advantageous features of a guidewire, especially its maneuverability and relatively small external diameter when compared with catheter systems typically utilized for fluid delivery procedures. The guidewire fluid delivery system includes a through lumen that is in fluid-passing communication with a passageway that is generally co-extensive with an elongated coil component of the guidewire system. Fluid passing through the through lumen exits the guidewire fluid delivery system at any number of selected delivery locations, typically along the coil, which can include at the distal tip of the guidewire.

During the course of various medical treatments and procedures, it is essential or desirable to deliver fluids to specific locations within the body. Such procedures are, in general, well known, and a variety of catheter devices are available for achieving fluid delivery in this regard. Typically, devices to accomplish these types of fluid delivery procedures require a system that combines a steerable guidewire and a separate fluid-delivery catheter that is guided in place by the steerable guidewire.

More specifically, a typical fluid delivery procedure incorporates the use of a generally solid guidewire in combination with the use of a fluid-delivery catheter. A guidewire is a device having excellent steerability and has a particularly narrow diameter, thereby facilitating its insertion into body passageways. Guidewires are generally designed to be maneuverable through narrow, tortuous and/or branching body passageways. Once inserted to the desired location, a guidewire provides the track over which a catheter then passes until the catheter is positioned along a pathway that is virtually the same as that of the inserted guidewire. In essence, the lumen of the catheter is threaded over the guidewire. Typically, the catheter lumen also serves as the passageway for delivery of fluids to the desired location within the body. Accordingly, before fluid delivery can actually occur, it is necessary to carefully remove the guidewire from the body and hence from out of the lumen of the fluid-delivery catheter.

This type of fluid delivery system accordingly requires three basic steps: insertion or implantation of the steerable guidewire, passage of the fluid-delivery catheter over the inserted guidewire, and removal of the guidewire from out of the lumen of the thus implanted or inserted catheter. The catheter is then ready for fluid delivery therethrough. Each of these steps must be done carefully and requires a noticeable amount of time, even for the most skilled surgeon. In addition, because the fluid-delivery catheter must fit over the steerable guidewire, the profile of the combination delivery system is significantly larger than the external diameter or profile of the steerable guidewire itself. Because of this, these types of combination systems are limited in their applications. They are generally not suitable for very narrow passageways, such as remote vessel locations and locations within the brain.

Accordingly, there is a need for a medical device fluid delivery system that has an especially small diameter to enable it to safely pass to and/or through very narrow and/or particularly delicate locations. It would also be advantageous to avoid a multiple-step insertion procedure in favor of a procedure whereby fluid delivery is accomplished directly by a device having the properties of a steerable guidewire. In other instances, it is advantageous that such a fluid-delivery steerable guidewire be used in association with a catheter positioned thereover in an arrangement in which the catheter performs a specific function, such as an angioplasty procedure, while the steerable guidewire remains in place and delivers needed fluids, such as blood or blood components, to a location distal of the lesion or the like being treated during an angioplasty procedure or the like. It would also be useful if such a steerable guidewire system could be provided which has the ability to distally remove fluids from a location within the body.

In summary, the perfusion guidewire achieves these objectives and provides advantageous results along these lines by providing a guidewire having the capability to pass fluid therethrough. The elongated medical device guidewire having fluid delivery and removal capabilities combines an elongated corewire surrounded at least in part by an elongated coil. A tubing member or sleeve generally covers the elongated coil and is made of a material which prevents passage of fluid, particularly liquids, therethrough, except at a designated location or at designated locations which can include an area of the coil that is distal of the distal edge of the tubing member. Through the use of an appropriate handle assembly, a fluid can be delivered into the fluid-delivery guidewire whereby the fluid flows between the elongated corewire and the tubing member or sleeve and generally along the elongated coil until reaching the delivery location or locations at which the fluid passes out of the fluid-delivery guidewire.

### Delivery Catheters

Delivery catheters are generally characterized by an elongate polymeric tube having a lumen extending along their length with at least one distally disposed port for local delivery of therapeutic or diagnostic agents. Such agents may be fluids, such as thrombolytic agents or radiopaque dyes, or devices, such as wires or vaso-occlusive coils or stents. Delivery catheters may be designed to track over a wire or designed to follow a blood flow to a desired treatment site.

Some delivery catheters slideably track over an independent wire rail to a distally remote location. A steerable guide wire is extended to a point at or near the desired treatment site. The delivery catheter tracks over the wire so that it may be pushed at its proximal end and be advanced along the wire rail to the desired site. Thus, in “over-the-wire” delivery catheter designs, the catheter need not perform in the sub-selection and independent advancement into distal tortuous anatomy. The guidewire has that function. Rather, the over-the-wire delivery catheter design may be optimized for delivery of the agent. For instance, such a structure may result in the potential for a larger lumen diameter for a given delivery catheter diameter.

An alternative method of placing a delivery catheter at a remote site is by allowing blood flow, rather than a guide wire, to direct the delivery catheter through the branching anatomy to a remote site. Such a method is generally available when such a catheter possesses the appropriate distal flexibility and composition to be influenced in its direction by the forces of physiological flow.

Once any of these catheters is placed at the desired site, drugs or other agents may be delivered through a distal port. Agents may be so delivered either through the co-axial space between the catheter and the wire or through the open lumen with the wire removed. Alternatively or in addition to any end holes for such fluid or device delivery, “side hole” delivery catheters may have one or more ports near to the catheter distal end. These ports may be in fluid communication either with a single lumen or with more than one lumen. These multiple distal ports are often in a desired spatial arrangement, such as at a pre-determined spaced interval longitudinally along the catheter axis, in a spiral arrangement, etc.

One example of a delivery catheter that tracks over a guidewire and is used for end hole fluid delivery is disclosed in U.S. Pat. No. 4,739,768 to Engelson. Engelson discloses a catheter which can be guided over a guide wire along a tortuous path of at least about 5 cm through vessels of less than about 3 mm lumen inner diameter. Engelson further discloses delivery of fluid materials through a lumen provided after the guide wire is withdrawn. The fluid materials may include radiopaque agents, vaso-occlusive agents, and pharmacological agents.

One example of a delivery catheter that is flow directed to a distal site and is used for end hole delivery of agents is disclosed in U.S. Pat. No. 5,336,205 to Zenzen. Zenzen discloses an elongate tubular body comprised of a relatively stiff tapered proximal segment and a relatively flexible and strong distal segment that may be directed to the target site by means of blood flow to the site. Once at the site, diagnostic, therapeutic, or vaso-occlusive agents may be infused through a catheter lumen provided and into the target site.

Although flow-directed devices such as the delivery device disclosed in Zenzen are designed to allow physiological flow to provide desired distal placement, such flow may, in certain cases, direct the device away from a desired site. This may be the case where there is a bifurcated vascular tree wherein a disproportionate division of flow exists at a bifurcation in branching vessels. When there is a higher rate of flow into one branch than the other, a flow directed catheter may more than likely follow that flow into the first vessel. However, the second vessel with the lower flow contribution from the feeding vessel may actually be the desired site for the delivery catheter.

There is a need for a catheter assembly that allows for a flow directed catheter to be positioned beyond a bifurcation and into a branch of the feeding vessel that has inferior physiological flow to another branch at the bifurcation.

### Balloon/Delivery Catheters

Guidewires have been disclosed having a fluid delivery port at or near its tip. Such a port may be located distally of the balloon, for instance through a guidewire. Additionally, balloon catheters have been disclosed having lumens ending in side ports located proximally of the balloon. Balloon catheters of the types just described are herein referred to as “balloon/delivery” catheters, although particular references may use different terminology.

One example of a dilation-drug delivery catheter is disclosed in U.S. Pat. No. 5,415,636 to Forman. Forman describes a dilation-drug delivery catheter having a dilation portion for dilating a stenosis and a drug delivery portion for delivering antithrombolytic, antiproliferative, or other medication to the dilation site. The drug delivery portion of the catheter is located within the dilation portion, which dilation portion can be retracted to reveal the drug delivery portion distal thereto after dilation. Occlusion balloons described in the reference are preferably provided on the drug delivery portion to isolate the dilation site during drug delivery. A dilatation lumen, a drug delivery lumen, a guide wire lumen, and an inflation lumen in an inner catheter shaft are provided in the catheter described.

Another example of a balloon catheter having a lumen connected to a port proximal of a balloon is disclosed in U.S. Pat. No. 5,413,581 to Goy. Goy discloses a balloon dilatation catheter having a first lumen extending along the entire length of a shaft, which lumen is connected to a pump and, at the distal end of the catheter, to the inside of the balloon. Through this lumen also passes a support wire connected firmly to the catheter at the catheter's distal end. The catheter shaft has an additional lumen which opens outwards of the catheter via an opening behind the proximal end of the balloon. Goy discloses that a controllable guide wire can be introduced into this additional lumen via an attachment piece, and that a measuring apparatus or an apparatus for introducing a contrast medium or drug can as well be connected to this additional lumen.

Another dilatation balloon catheter having an infusion lumen is found in U.S. Pat. No. 5,368,567 to Lee. Lee discloses a dilatation catheter having two or more associated fluid carrying tubes, the operative or distal end of one of which supplies fluid to inflate an expansible balloon. The operable or distal end of the other tube supplies an injectable dye or contrast enhancing fluid adjacent the proximal end of the balloon. The catheter embodiments disclosed by Lee also include a guide wire lumen separate from the balloon inflation lumen. The proximal end of the short guide wire lumen preferably begins adjacent the distal opening of a hollow tube lumen for injecting contrast medium proximal of the balloon. The distal end of the short guide wire lumen terminates distally of the sealed balloon.

U.S. Pat. No. 4,983,166 to Yamawaki discloses a balloon catheter having a balloon and a main passage ending in an opening behind the balloon. The reference discloses that, with the balloon inflated, drugs may be delivered through the opening and into other branches than that in which the balloon catheter tip is placed. Yamawaki discloses use of a circulatory curved tip end portion of the balloon catheter for inserting the catheter from a wider artery into a narrower artery diverging from the wider artery at an acute angle. The reference further discloses that a guidewire may be placed in the drug delivery passageway and out the opening behind the balloon, but does not otherwise disclose a guidewire lumen for tracking of the catheter over a guidewire to a remote in-vivo location.

One example of a medical treatment that has, for certain applications, been facilitated by delivery of therapeutic treatments through balloon/delivery catheters is artificial vaso-occlusion. Artificial vaso-occlusion or embolization is a medical treatment that often involves locally delivering a vaso-occlusive agent to a desired site. The agent therein causes a physiological occlusive response to flow or otherwise blocks or fills a body space. Different sites in the body where vaso-occlusion treatments have been used include aneurysms, blood vessels, and arteriovenous malformations.

Examples of various chemicals that have been used for in-vivo artificial vaso-occlusion include ethanol, estrogen, polyvinyl acetate (“PVA”), ethylene vinyl alcohol (“EVAL”), cellulose acetate polymer, or combinations thereof. Known delivery techniques for such vaso-occlusive agents include delivery through microcatheter-type delivery catheters, and delivery through balloon/delivery catheters having delivery ports adjacent to expandable balloons.

Accurate placement during the delivery of vaso-occlusive agents is critical, since inaccurate placement of the occlusive device or agent may undesirably occlude regions where continued flow must or should be maintained. Appropriate placement is especially important where an agent is relatively fluid and may migrate from the desired site if exposed to physiological flow. Therefore it is often desirable to isolate target delivery sites from flow into or from adjacent vasculature.

One reference to arterial embolization through a balloon catheter with a passage ending in an opening proximal of the balloon is in U.S. Pat. No. 4,983,166 to Yamawaki (introduced above). Yamawaki discloses use of a circulatory curved tip on the balloon catheter for subselecting of side branches.

Another example of an embolization treatment via balloon catheter delivery of chemical embolizing agents in the renal arteries is disclosed in “Nonsurgical Treatment of AVM: Development of New Liquid Embolization Method,” Takahashi, et al., Suzuki J., ed., Advances in surgery for cerebral stroke, Tokyo, Japan: Springer-Verlag 1988:215-224. Takahashi discloses percutaneous delivery of conjugated estrogen diluted in 25% ethanol and polyvinyl acetate (“PVac”). According to the disclosure, PVac, when diluted in alcohol, becomes gelatinous in one second upon contacting water. Disclosed treatment methods included injections of PVac during proximal occlusion using slow leaking double lumen balloon catheters after 20 minute infusion of alcohol.

These balloon/delivery catheter references disclose vaso-occlusive fluid delivery through a delivery catheter port, together with isolation of a vascular site by means of an expandable balloon integrated with the delivery catheter. However, vasculatures and disease/injury states vary among patients. The desired spatial arrangement of an occlusive balloon and a delivery port may vary accordingly for a given vaso-occlusion procedure. There is a need for a device assembly that allows for adjustable positioning of an occlusive balloon relative to a delivery port of a delivery catheter.

In addition to delivery of fluids for vaso-occlusion, a more recent artificial vaso-occlusion technique involves the delivery of implantable devices, particularly vaso-occlusive coils, to the desired site of occlusion. Such coils generally are made of a metal or metal alloy and may have various primary and secondary winds and dimensions. One of the benefits of using vaso-occlusive coils over other techniques is that a natural expansion of the coil diameter or other mechanical mechanism may be imparted to the coil such that it readily anchors itself against the walls of the delivery site. This may occur, for example, upon deployment of the coil from a restrained, stretched state within a catheter lumen and into a less constraining body space such that the coil's geometry passively transforms back to its relaxed, unrestrained memory state—or at least until it encounters a vessel wall against which it exerts a force to complete the anchoring process.

In general, vaso-occlusive coils are delivered through microcatheters such as the type disclosed in U.S. Pat. No. 4,739,768 to Engelson (previously discussed). The microcatheter tracks a guide wire to a point just proximal of or within the desired site for occlusion. The coil is advanced through the microcatheter and out the distal end hole and is thereby implanted into the adjacent space. The mechanisms chosen for advancing the coil through and out of the delivery catheter and the resultant coil designs may vary. Coils that are mechanically detached from an integrated pusher after exiting a delivery catheter have been disclosed for example in U.S. Pat. No. 5,261,916 to Engelson. Similarly, electrically detachable coils have been disclosed for example in U.S. Pat. No. 5,122,136 to Guglielmi, et al. Pushable coils have also been disclosed, for instance in U.S. Pat. No. 4,994,069 to Ritchart, et al. Additionally, pending U.S. patent application Ser. No. 08/413,970, filed Mar. 30, 1995 describes very soft and flexible coils which are flow-injectable through the delivery catheter using, e.g., saline solution.

Although each type of previously disclosed coil may have distinct benefits, there remain certain clinical challenges. For example, one such challenge is the occasional occurrence of a recoil phenomena at the distal end of a delivery catheter when a coil implant is discharged. This recoil phenomena may compromise the ability accurately to place the coil or coils where desired.

One artificial vaso-occlusion treatment where the recoil phenomena is particularly undesirable is during vaso-occlusive coil delivery into aneurysms, such as berry aneurysms, that are formed along blood vessel axes. These coils may be delivered to the aneurysm in the following manner. A microcatheter is steered into or adjacent the entrance of an aneurysm, aided by a steerable wire. The wire is then withdrawn from the microcatheter lumen and replaced by the vaso-occlusion coil. The vaso-occlusion coil is advanced through and out of the microcatheter, desirably being completely delivered into the aneurysm. Where recoil forces are experienced at the microcatheter tip, the tip may unseat from its positioning in the aneurysm. A portion of the coil might then trail out of the aneurysm entrance zone and into the feeding vessel. This may cause undesirable occlusive response in the good, feeding vessel. Also, there may be an increased risk that the blood flow may induce movement of the coil farther out of the aneurysm, resulting in a more developed embolus in the good vessel.

Although catheter distal tip shapes may be formed on delivery microcatheters to help support the distal tip during deployment of vaso-occlusive agents, this may in some circumstances provide only a partial solution. There is a need for a delivery catheter assembly that prevents or minimizes delivery catheter recoil during delivery of vaso-occlusive agents.

None of the cited references disclose a balloon/delivery catheter assembly having a balloon catheter with an expandable balloon that is variably positionable along the polymeric delivery shaft of an over-the-wire or flow-directable delivery catheter.

Nor do these references disclose a balloon/delivery catheter assembly that includes a single-lumen balloon catheter having a polymeric delivery catheter shaft coaxially disposed within the inflation lumen thereof, such coaxial arrangement providing a tightly toleranced distal fluid seal region that allows for controllably sustained balloon inflation.

Nor do these references disclose a balloon/delivery catheter assembly that allows for a balloon to be advanced over and distally beyond a flow-directable catheter in a first branch of a bifurcation, such that subsequent inflation of the balloon diverts flow into a second branch of the bifurcation sufficient to reposition the flow-directed catheter into the second branch.

Nor do these references disclose a balloon/delivery catheter assembly that minimizes recoil to the delivery catheter during delivery of agents through a delivery catheter port by allowing variable positioning and inflating of a balloon at a chosen location along a delivery catheter shaft.

### SUMMARY OF THE INVENTION

This invention is a catheter assembly in which the proximal end is of a non-compliant inner and outer diameter and the distal end is expandable and collapsible. The proximal shaft of the catheter has a delivery lumen extending between a proximal delivery port and the distal portion. The distal catheter end portion is expandable and collapsible along a portion of the delivery shaft that provides a continuous lumen with the proximal portion.

In one embodiment of the assembly described, the proximal shaft has a longitudinal lumen of fixed radius and length and a distal portion that expands and collapses radially. Preferable to this embodiment, the distal catheter lumen expands with internal shaft pressure returns to approximately its original radial dimension upon pressure release. Potential sources of internal pressurization include fluid injection from proximal end or a device, such as a vaso-occlusive coil, stent or guidewire, being pushed through lumen. In an alternative distal end embodiment, the shaft remains in its expandable position upon internal pressure release. Further, there may be a valve seat in a distal end or a rigid or semi-rigid structure built into the wall and/or lumen to maintain the expanded position. In another alternative distal end embodiment, the distal end may be expanded by a non-pressurized means, such as with cables or wires that are controlled at the proximal end or portion of the catheter.

Also contemplated are preferred methods of using the embodiments described. The catheter may be desirably placed at a chosen position without the use of a guide wire and then the fluid or device can be introduced into the body. Alternatively, the delivery catheter may be introduced into the body to the desired location by tracking over a guidewire or an alternative guide device or system, such as an external magnet. In a further aspect of the invention, the catheter may be remain is the chosen location for a short duration (i.e., less than eight hours), such as when used for a vascular intervention. It may also remain permanently or for an extended duration (i.e., greater than eight hours), such as procedures requiring local drug delivery or any other diagnostic or therapeutic agent.

In one medical procedure, the catheter may be advanced to a lesion and then used to deliver diagnostic or therapeutic agents or devices and removed shortly thereafter. Or, the catheter may be placed in a preferred location for an extended period of time for the purpose of delivering a drug or other agent for the purpose of monitoring a condition.

What is claim is:

1. A catheter assembly comprising of:
a hub with external communicating port;
a non-compliant proximal shaft section with a length between 1.0-200 centimeters;
said proximal shaft section attached to said hub;
said proximal shaft section has an outer diameter between 0.3 mm-3.8 mm and inner lumen between 0.2 mm-3.5 mm;
said proximal shaft section is comprised of a polymeric inner liner, at least one polymeric outer layer, and metallic or rigid polymeric structural component between said inner and outer layers;
a radially expandable and collapsible distal shaft section distal of said proximal shaft section;
said distal shaft section has an outer diameter in the range of 0.15 mm-3.5 mm and inner diameter between 0.0 mm-3.2 mm in its collapsed position;
said distal shaft section length between 0.75 cm-200 cm;
said distal shaft comprises at least three separate materials: a polymeric inner liner, at least one outer layer of polymeric material, and a structural element or elements between outer and inner layers or imbedded therein;
said distal shaft section is more flexible than said proximal section.
an atraumatic expandable and collapsible tip extending distally of said distal shaft section that is comprised of at least one polymer.
an external communicating port on distal end of said tip;
a lumen extending from proximal external communicating port to a distal external communicating port;

2. The assembly of claim 1, wherein said distal shaft section inner liner is comprised of a fluoropolymer or polyethylene.

3. The assembly of claim 1, wherein said distal shaft section inner liner lumen has a hydrophilic or hydrophobic coating.

4. The assembly of claim 1, wherein said distal shaft section inner liner is impregnated or imbedded with hydrophilic or hydrophobic material.

5. The assembly of claim 1, wherein said distal shaft section structural component is a slotted tube.

6. The assembly of claim 1, wherein said distal shaft section structural component consists of multiple interconnected segments.

7. The assembly of claim 1, wherein said distal shaft section structural component consists of axially striped materials.

8. The assembly of claim 1, wherein said distal outer layer consists of a compliant polymer.

9. The assembly of claim 1, wherein said distal outer layer consists of multiple polymers, one being significantly more rigid, the other a compliant polymer.

10. The assembly of claim 1, wherein said distal shaft is comprised of at least one layer of a folded polymer.

11. The assembly of claim 1, wherein tip is flared.

12. The assembly of claim 1, wherein tip is tapered.

13. The assembly of claim 1, wherein tip is of uniform diameter.

14. The assembly of claim 1, wherein said distal section is expanded by changes in internal pressure, such as those caused by a fluid injected into proximal end or by passing a device through lumen.

15. The assembly of claim 1, wherein said distal section is expanded or collapsed by application of pull wires controlled from proximal end, or by a magnetic, chemical or electric signal applied to structural component(s).

16. The assembly of claim 1, wherein said distal section is expanded via fluid force substantially directed from distal to proximal port.

17. A method for using a collapsible and expandable catheter including the following steps:
providing a guidewire and collapsible and expandable catheter;
advancing the guidewire to the target site;
advancing the catheter over the guidewire to the target site;
removing the guidewire;
delivering a fluid and/or device from the proximal to distal end, or drawing a fluid and/or solids into the distal end of the catheter by producing fluid flow substantially directed from the distal to proximal port of catheter.

18. A method for using collapsible and expandable catheter including the following steps:
providing a collapsible and expandable catheter;
advancing the catheter to the target site;
delivering a fluid and/or device from the proximal to distal end, or drawing a fluid and/or solids into the distal end of the catheter by producing fluid flow substantially directed from the distal to the proximal port of catheter.
